(19) United States
(12) Patent Application Publication
(10) Pub. No.: US 2013/0116756 A1
(43) Pub. Date: May 9, 2013

(54) UV-A OR UV-B-EMITTING DISCHARGE LAMP

(71) Applicants: Thomas Juestel, Witten (DE); Georg Greuel, Roetgen (DE)

(72) Inventors: Thomas Juestel, Witten (DE); Georg Greuel, Roetgen (DE)

(73) Assignee: KONINKLIJKE PHILIPS ELECTRONICS N.V., EINDHOVEN (NL)

(21) Appl. No.: 13/809,521

(22) PCT Filed: Jul. 8, 2011
(86) PCT No.: PCT/IB2011/053045

§ 371 Date: Jan. 10, 2013

(30) Foreign Application Priority Data
Jul. 13, 2010 (EP) 10169377.8

(51) Int. Cl. H01J 61/44; A61N 5/06

(52) U.S. Cl. CPC H01J 61/44; A61N 5/0614; A61N 5/0616
USPC 607/88; 313/487; 422/186.3

(57) ABSTRACT

The invention relates to a discharge lamp with a novel garnet-type Pr or Tm doped phosphor with excellent UV-A and UV-B spectral properties.

### FIELD OF THE INVENTION

The present invention is directed to novel materials for light emitting devices, especially to the field of novel materials for discharge lamps emitting UV radiation.

### BACKGROUND OF THE INVENTION

Fluorescent lamps which comprise an UV emitting phosphor are widely applied for cosmetic and medical purposes. These lamps usually generate UV light by e.g. utilizing an Hg low-pressure discharge and a luminescent screen comprising UV-B or UV-A phosphors or a blend of several UV-A/B phosphors. The most commonly applied phosphors are LaPO₄:Ce, SrAl₁₂O₁₉:Ce,Na, or LaB₃O₆:Bi,Gd as UV-B emitter and (Y,Gd)PO₄:Ce, BaSi₂O₅:Pb, or SrB₄O₇:Eu as UV-A emitter.

However, Hg discharge lamps for disinfection or purification purposes do not use a luminescent screen, since the spectral power distribution of the emission spectrum of a low-or medium-pressure Hg discharge nicely coincides with the GAC and with the absorption edge of molecular Oxygen and water.

The main drawback of the presently applied low- and medium-pressure Hg discharge lamps is their strong dependence on the radiation output from the (water, air) temperature. This is caused by the vapor pressure of liquid Hg, which is a sensitive function of temperature.

Moreover, chemical interaction between the Hg discharge and the glass vessel causes the formation of color centers, which in turn causes an efficiency reduction. Formation of an absorbing layer due to color centers is reduced, once nanoscale particles or phosphor particles are coated on top of the glass surface. Therefore, low- and medium-pressure Hg discharge lamps show strong degradation and must be replaced after about 1000 h operation.

Therefore, there is the need for alternative phosphors, especially for UV-A and/or UV-B lamps, which at least partly overcome the above-mentioned drawbacks and which have a longer lifetime.

### SUMMARY OF THE INVENTION

It is an object of the present invention to provide a discharge lamp which is at least partly able to overcome the above-mentioned drawbacks, and especially to enable building a discharge lamp with good or improved lighting features together with an increased lifetime for a wide range of applications.

This object is achieved by a discharge lamp according to claim 1 of the present invention. Accordingly, a discharge lamp, preferably a low-pressure gas discharge lamp, is provided, wherein said discharge lamp is provided with a discharge vessel comprising a gas filling with a discharge-maintaining composition, wherein at least a part of a wall of the discharge vessel is provided with a luminescent material comprising Lu_{1-x-y-a-b}YₓGd_{y})₃Al_{5-z}Ga_{z}O₁₂:RE1ₐ:RE2_{b}, with RE1 being selected out of the group comprising Pr³⁺, Tm³⁺ or mixtures thereof, RE2 being selected out of the group comprising Nd³⁺, Sm³⁺, Eu³⁺, Tb³⁺, Dy³⁺, Ho³⁺, Er³⁺ or mixtures thereof and a is >0 and ≦0.1, b is ≧0 and ≦0.1, x is ≧0 and ≦1, y is ≧0 and ≦1 (with the proviso that a+b+x+y are ≦1) and z is ≧0 and ≦5.

Surprisingly, it has been found that, for a wide range of applications within the present invention, such a discharge lamp has at least one of the following advantages:

a higher lamp efficacy due to the optimized emission spectrum to the action curve of the application and due to less re-absorption by the phosphor layer,

a higher stability of the UV output and thus a higher operational lifetime of the UV light source

the ease of application of fast switching cycles and the fast lamp run-up time

an adjustable UV emission spectrum

and a high flexibility of the lamp with respect to its geometry and switching

Preferably the discharge lamp is a Xe, Ne, or Xe/Ne excimer discharge lamp.

According to a preferred embodiment of the present invention, especially if RE1 comprises Pr, then z is ≧0.5 and ≦5, i.e. the luminescent material comprises Praseodymium and Gallium. Surprisingly, the inventors have found out that, as a result thereof, a blue-shift emission of the resulting luminescent material can be observed which leads to luminescent materials which are highly desirable for many applications within the present invention. This effect can be observed for many applications with Tm as well, although for Pr it is somewhat more dominant. However, also embodiments are preferred where RE1 comprises Tm and z is ≧0.5 and ≦5.

According to a preferred embodiment of the present invention z is ≧1 and ≦3.

According to a further preferred embodiment, at least a part of a wall of the discharge vessel is provided with a basic metal oxide, preferably selected out of the group comprising MgO, MgAl₂O₄, Al₂O₃, or Ln₂O₃ (Ln=Sc, Y, La, Gd, Lu) or mixtures thereof. This has been shown to be advantageous for many applications within the present invention due to the observed reduction of the decrease in radiation output over the lifetime.

The basic metal oxide is preferably provided as a nanoscale particle material with an average particle size of ≧10 nm and ≦1 μm, preferably ≧100 nm and ≦500 nm. Preferably, it is provided either together with the luminescent material (e.g. a layer is formed comprising both materials) or as a layer on top of or underneath the luminescent material.

According to a preferred embodiment, at least a part of a wall of the discharge vessel is provided with a further luminescent material selected out of the group comprising (Y₁₋ₓ₋ₐLuₓ)BO₃:Prₐ, (Y₁₋ₓ₋ₐLuₓ)PO₄:Prₐ, (Y₁₋ₓ₋ₐLuₓ)PO₄:Biₐ, La₁₋ₐPO₄:Prₐ, (Y₁₋ₓ₋ₐLuₓ)AlO₃:Prₐ, (Ca₁₋ₓ₋₂ₐSrₓ)₂P₂O₇:PrₐNaₐ, (Ca₁₋ₓ₋₂ₐSrₓ)Al₁₂O₁₉:CeₐNaₐ, or (La₁₋ₓ₋ₐGdₓ)PO₄:Ceₐ with x≧0 and ≦1 and a is >0 and ≦0.1 (for all materials) or mixtures thereof.

By virtue thereof, for many applications an optimization of the lamp spectrum to the action spectrum of the given application is feasible.

The present invention furthermore relates to the use of (Lu_{1-x-y-a-b}YₓGd_{y})₃Al_{5-z}Ga_{z}O₁₂:RE1ₐ:RE2_{b}, with RE1 being selected out of the group comprising Pr³⁺, Tm³⁺ or mixtures thereof, RE2 being selected out of the group comprising Nd³⁺, Sm³⁺, Eu³⁺, Tb³⁺, Dy³⁺, Ho³⁺, Er³⁺ or mixtures thereof and a is >0 and ≦0.1, b is ≧0 and ≦0.1, x is ≧0 and ≦1, y is ≧0 and ≦1 (with the proviso that a+b+x+y are ≦1) and z is ≧0 and ≦5 as an activator in UV-A and/or UV-B emitting illumination systems.

A system comprising a discharge lamp as described herein or making use of the phosphor material as described above may be used in one or more of the following applications:

Equipment for medical therapy (e.g. Treatment of skin diseases such as Psoriasis)

Equipment for cosmetic skin treatment (e.g. tanning devices)

Water sterilization and/or purification applications, e.g. by using an additional TiO₂ photocatalyst

Chemical reactors, e.g. for the photochemical synthesis of advanced chemical products, e.g. Vitamin D₃

The aforementioned components, as well as the claimed components and the components to be used in accordance with the invention in the described embodiments, are not subject to any special exceptions with respect to their size, shape, Discharge lamp selection and technical concept, so that the selection criteria known in the pertinent field can be applied without limitations.

### BRIEF DESCRIPTION OF THE DRAWINGS

Additional details, features, characteristics and advantages of the object of the invention are disclosed in the subclaims, the Figures and the following description of the respective Figures and examples, which—in exemplary fashion—show several embodiments and examples of discharge lamps according to the invention.

FIG. 1 shows a very schematic cross-sectional view of a discharge lamp according to a first embodiment of the present invention.

FIG. 2 shows an emission spectrum of a first luminescent material according to the present invention (Example I).

FIG. 3 shows the excitation spectrum of the material of FIG. 2.

FIG. 4 shows an emission spectrum of a second luminescent material according to the present invention (Example II).

FIG. 5 shows the excitation spectrum of the material of FIG. 4.

FIG. 6 shows an emission spectrum of a second luminescent material according to the present invention (Example III).

FIG. 7 shows the excitation spectrum of the material of FIG. 6.

FIG. 8 shows the emission spectrum of a Xe excimer discharge lamp comprising the materials of Examples I and II.

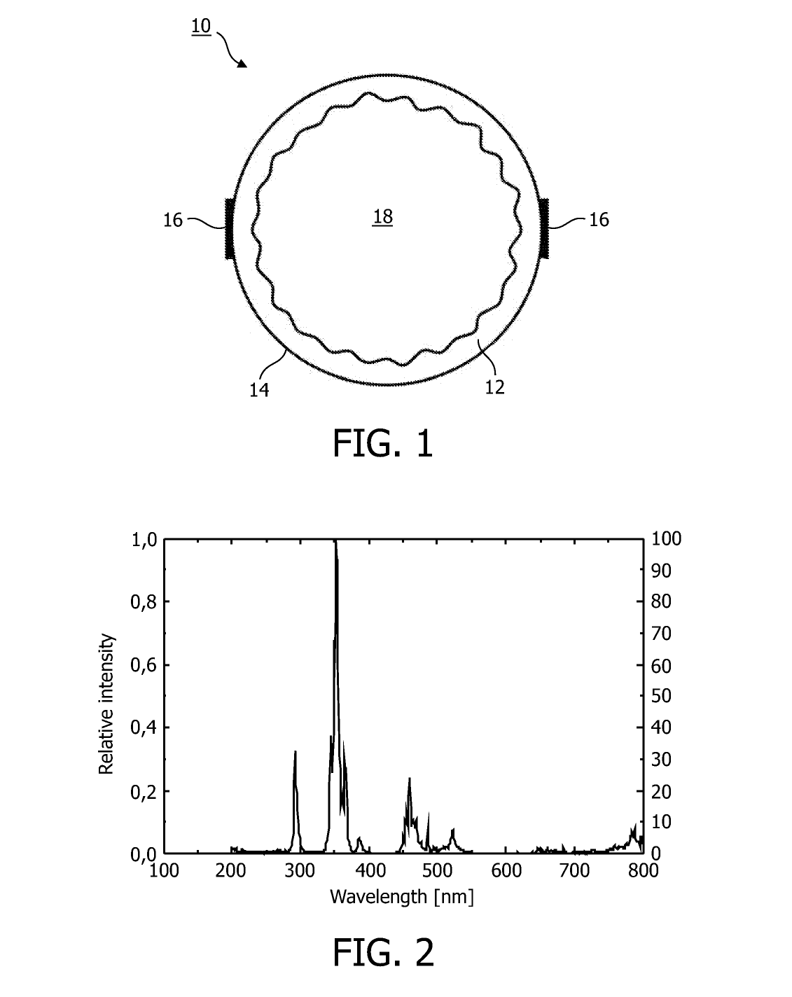

FIG. 1 shows a very schematic cross-sectional view of a discharge lamp according to a first embodiment of the present invention. The discharge lamp 10 (which is principally prior art) comprises a glass tube 14 in which a phosphor 12 is provided. This phosphor comprises the luminescent material of the present invention. Depending on the application also further phosphors and basic metal oxide nanoparticle may be present in this area. Furthermore two electrodes (e.g. made of Al) 18 are provided.

The invention will furthermore be understood by the following Inventive Example which is merely for illustration of the invention only and non-limiting.

### EXAMPLE I

Example I refers to Lu₃Al₅O₁₂:Tm, which was made in the following way:

The starting materials 59.093 g (148.5 mmol) Lu₂O₃, 24.471 g (240 mmol) Al₂O₃ and 1.014 g (3.0 mmol) Tm₂O₃ are dissolved in conc. HNO₃. Then the solvent is removed by evaporation and the remaining powder is fired for 2 h at 600° C. to decompose the nitrates.

Subsequently, the obtained is powderized and 1.680 g (20 mmol) AlF₃ are added as a flux. Afterwards, the powder is annealed for 2 h at 1300° C. in a CO atmosphere, powderized and fired again for 4 h between 1500 and 1700° C. in a CO atmosphere. Finally, the obtained powder cake is crushed and the powder is sieved through a 36 μm sieve.

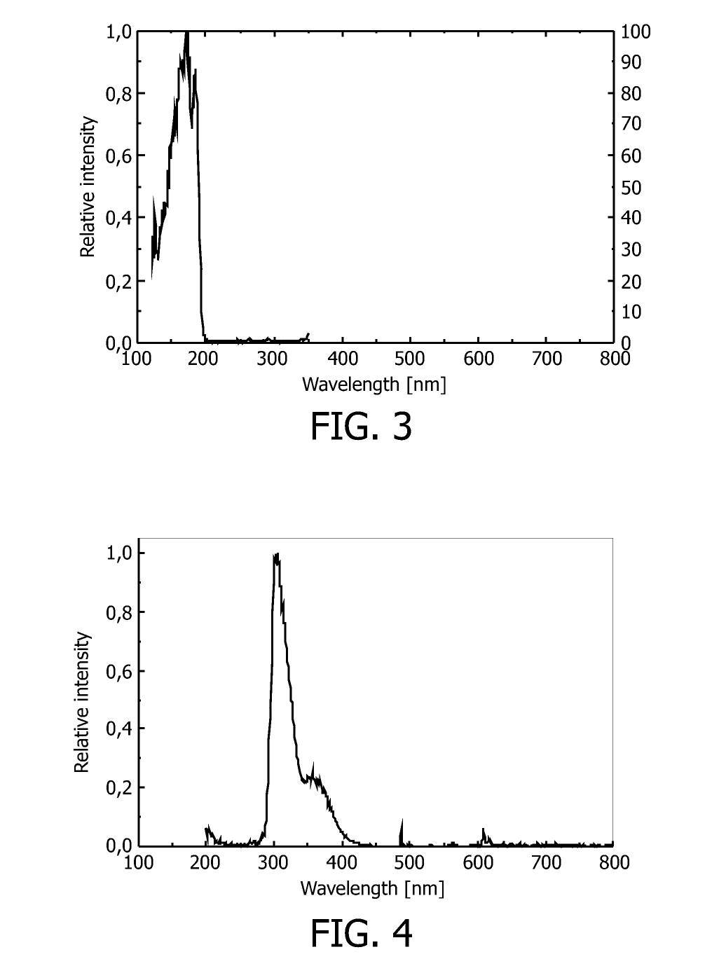

FIG. 2 shows the emission spectrum, FIG. 3 shows the excitation spectrum of the material of Example I. It can clearly be seen that this material is an excellent material for use in discharge lamps for UV-A radiation

### EXAMPLE II

Example II refers to Lu₃GaAl₄O₁₂:Pr, which was made in the following way: The starting materials 59.093 g (148.5 mmol) Lu₂O₃, 19.577 g (192 mmol) Al₂O₃, 8.997 g (48 mmol) Ga₂O₃, and 3.064 g (3.0 mmol) Pr₆O₁₁ are dissolved in conc. HNO₃. Then the solvent is removed by evaporation and the remaining powder is fired for 2 h at 600° C. to decompose the nitrates.

Subsequently, the obtained is powderized and 1.680 g (20 mmol) AlF₃ are added as a flux. Afterwards, the powder is annealed for 2 h at 1300° C. in a CO atmosphere, powderized and fired again for 4 h between 1500 and 1700° C. in a CO atmosphere. Finally, the obtained powder cake is crushed and the powder is sieved through a 36 μm sieve.

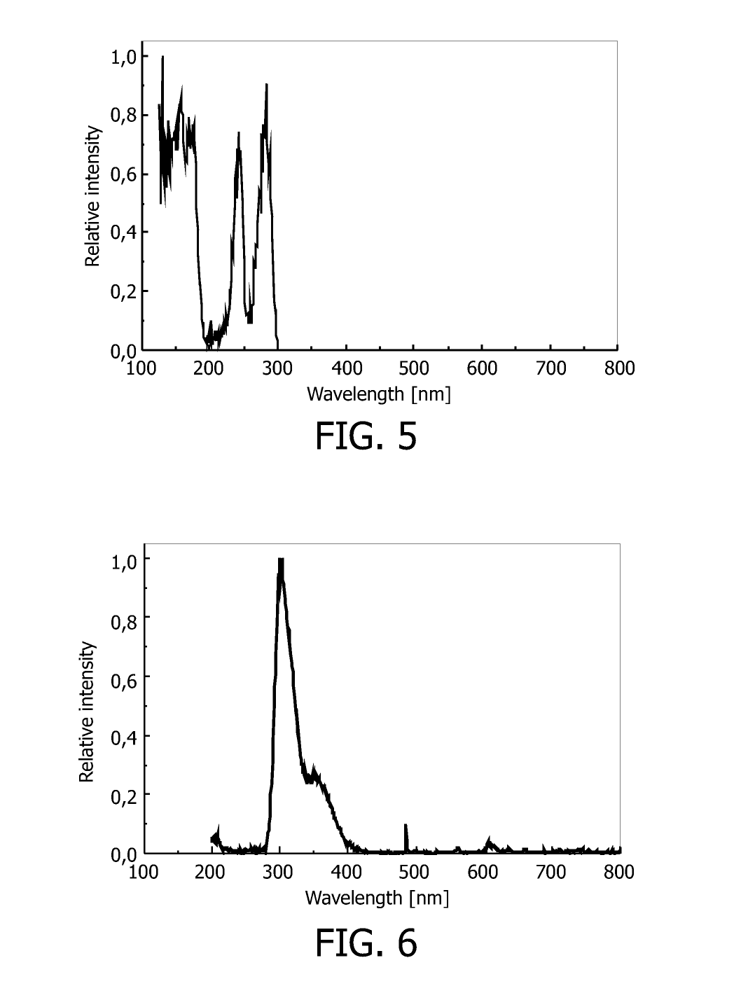

FIG. 4 shows the emission spectrum, FIG. 5 exhibits the excitation spectrum of the material of Example I. It is obvious that this material is an excellent material for use in discharge lamps for UV-B radiation.

### EXAMPLE III

Example III refers to Lu₃Ga₂Al₃O₁₂:Pr, which was made in analogous fashion to the material of Example II.

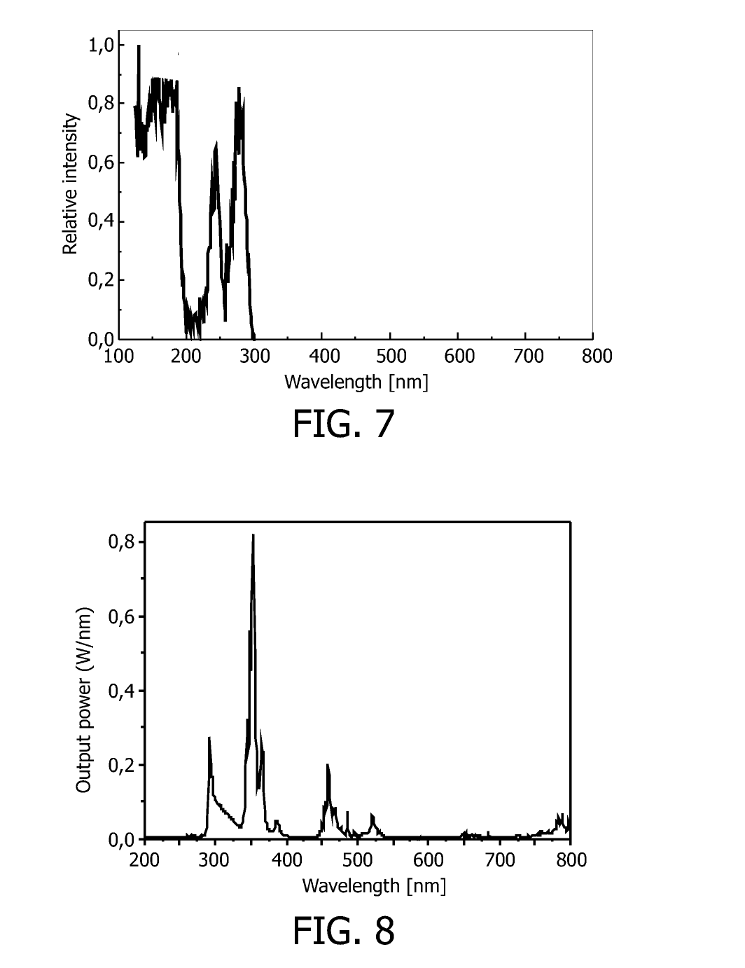

FIG. 6 shows the emission spectrum, FIG. 7 shows the excitation spectrum of the material of Example I. It can clearly be seen that this material is an excellent material for use in discharge lamps for UV-B radiation.

### EXEMPLARY XE EXCIMER DISCHARGE LAMP

The invention will furthermore be illustrated by means of an exemplary Xe excimer discharge lamp using the materials of Example I and II. This lamp was made in the following way:

A suspension of nanoparticles MgO is made on a butylacetate basis with nitrocellulose as a binder. The suspension is applied to the inner wall of a standard UV transparent glass tube by using a flow coat related procedure. Then, a suspension of a Lu₃GaAl₄O₁₂:Pr (Example II) and Lu₃Al₅O₁₂:Tm (Example I) is prepared on a butylacetate basis with nitrocellulose as a binder. By means of a similar flow coat related procedure, the suspension is applied to the inner wall of the precoated lamp tube with a typical phosphor layer weight in the range of 2-6 mg/cm². The binder is burned in a standard heating cycle with peak temperatures between 500 and 600° C. The glass tube is filled with Xe using a thorough pumping cycle, so as to strictly exclude Oxygen impurities, and finally sealed. Typical gas pressures are 200-300 mbar pure Xe. Al-electrodes are attached to the outer side of the tube by means of adhesion or painting. The lamps are typically operated at 5 kV and 25 kHz, using a pulse driving scheme. The emission spectrum is determined using an optical spectrum multianalyser and is shown in FIG. 8.

The particular combinations of elements and features in the above detailed embodiments are exemplary only; the interchanging and substitution of these teachings with other teachings in this patent and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. The invention's scope is defined in the following claims and equivalents thereto. Furthermore, reference signs used in the description and claims do not limit the scope of the invention as claimed.

1. Discharge lamp, provided with a discharge vessel comprising a gas filling with a discharge-maintaining composition, wherein at least a part of a wall of the discharge vessel is provided with a luminescent material comprising (Lu_{1-x-y-a-b}YₓGd_{y})₃Al_{5-z}Ga_{z}O₁₂ :RE1ₐ:RE2_{b}, with RE1 being Tm³⁺ or mixtures thereof with Pr³⁺, RE2 being selected out of the group comprising Nd³⁺, Sm³⁺, Eu³⁺, Tb³⁺, Dy³⁺, Ho³⁺, Er³⁺ or mixtures thereof and a is >0 and ≦0.1, b is ≧0 and ≦0.1, x is ≧0 and ≦1, y is ≧0 and ≦1 (with the proviso that a+b+x+y are ≦1) and z is ≧0 and ≦5.

2. The discharge lamp of claim 1, wherein the discharge lamp is a Xe, Ne, or Xe/Ne excimer discharge lamp.

3. The discharge lamp of claim 1, wherein z is ≧0.5 and ≦5.

4. The discharge lamp of claim 3, wherein z is ≧1 and ≦3.

5. The discharge lamp of claim 1, wherein at least a part of a wall of the discharge vessel is provided with a basic metal oxide, preferably selected out of the group MgO, MgAl₂O₄, Al₂O₃, or Ln₂O₃ (Ln=Sc, Y, La, Gd, Lu) or mixtures thereof.

6. The discharge lamp of claim 1, wherein at least a part of a wall of the discharge vessel is provided with a further luminescent material selected out of the group comprising (Y₁₋ₓ₋ₐLuₓ)BO₃:Prₐ, (Y₁₋ₓ₋ₐLuₓ)PO₄:Prₐ, (Y₁₋ₓ₋ₐLuₓ)PO₄:Biₐ, La₁₋ₐPO₄:Prₐ, (Y₁₋ₓ₋ₐLuₓ)AlO₃:Prₐ, (Ca₁₋ₓ₋₂ₐSrₓ)₂P₂O₇:PrₐNaₐ, (Ca₁₋ₓ₋₂ₐSrₓ)Al₁₂O₁₉:CeₐNaₐ, or (La₁₋ₓ₋ₐGdₓ)PO₄:Ceₐ with x≧0 and ≦1 and a is >0 and ≦0.1 (for all materials) or mixtures thereof.

7. Use of (Lu_{1-x-y-a-b}YₓGd_{y})₃Al_{5-z}Ga_{z}O₁₂:RE1ₐ:RE2_{b}, with RE1 being Tm³⁺ or mixtures thereof with Pr³⁺, RE2 being selected out of the group comprising Nd³⁺, Sm³⁺, Eu³⁺, Tb³⁺, Dy³⁺, Ho³⁺, Er³⁺ or mixtures thereof and a is >0 and ≦0.1, b is ≧0 and ≦0.1, x is ≧0 and ≦1, y is ≧0 and ≦1 (with the proviso that a+b+x+y are ≦1) and z is ≧0 and ≦5 as an activator in UV-A and/or UV-B emitting illumination systems.

8. A system comprising a discharge lamp according to claim 1, the system being used in one or more of the following applications:
Equipment for medical therapy (e.g. Treatment of skin diseases such as Psoriasis)
Equipment for cosmetic skin treatment (e.g. tanning devices)
Water sterilization and/or purification applications, e.g. by using an additional TiO₂ photocatalyst
Chemical reactors, e.g. for the photochemical synthesis of advanced chemical products, e.g. Vitamin D₃.
